(19) United States
(12) Patent Application Publication
(10) Pub. No.: US 2015/0060462 A1
(43) Pub. Date: Mar. 5, 2015

(54) PORTABLE NEUROTOXIN INJECTION TRAY AND ASSOCIATED APPARATUS, SYSTEM, AND METHOD

(71) Applicants: Rand Colbert, Cedar City, UT (US); Russell G. Olsen, Cedar City, UT (US)

(72) Inventors: Rand Colbert, Cedar City, UT (US); Russell G. Olsen, Cedar City, UT (US)

(21) Appl. No.: 14/477,844

(22) Filed: Sep. 4, 2014

Related U.S. Application Data
(60) Provisional application No. 61/873,449, filed on Sep. 4, 2013.
(60) Provisional application No. 62/016,768, filed on Jun. 25, 2014.

(51) Int. Cl. A61M 5/00; A61B 19/02; A61J 1/20; A61K 38/48

(52) U.S. Cl. CPC A61M 5/002; A61K 38/4893; C12Y 304/24069; A61B 19/0271; A61B 19/0287; A61J 1/2096; A61B 2019/0278; A61B 2019/0286; A61B 2019/0209; A61M 2205/3606; A61J 2001/201
USPC 220/553; 424/94.67

(57) ABSTRACT

A portable neurotoxin injection tray apparatus that includes a vial section with multiple compartments. Each compartment is capable of storing at least one neurotoxin vial or at least one saline vial. The apparatus also includes a dosage compartment with a tilted support member for positioning at least one of the neurotoxin vials in a tilted orientation. Further, the apparatus includes an injection syringe section that includes multiple compartments, where each compartment is designated to store at least one injection syringe filled with a different dosage of neurotoxin.

### CROSS-REFERENCE TO RELATED APPLICATIONS

This application claims the benefit of U.S. Provisional Patent Application No. 61/873,449, filed Sep. 4, 2013, and U.S. Provisional Patent Application No. 62/016,768, filed Jun. 25, 2014, which are incorporated herein by reference.

### FIELD

The present disclosure relates generally to pharmaceutical drugs, and more particularly to the storage, preparation, and administration of neurotoxins.

### BACKGROUND

Neurotoxins, such as botulinum toxin, or more commonly called BOTOX®, are used for certain medical and cosmetic procedures. Generally, the neurotoxins are manufactured and transported to a practitioner's office in a pre-application state within an industry-standard vial. Therefore, practitioners usually must prepare the neurotoxin for use by converting the toxin into a useable or deliverable state. Often, this preparation includes reconstituting a solid powder neurotoxin using a certain amount of saline solution. Once reconstituted, the neurotoxin must generally be used within a certain time frame to prevent degradation of the neurotoxin.

Conventional systems and methods for the preparation, storage, and delivery of neurotoxin treatments are time-consuming, wasteful, difficult, and potentially unsafe and confusing to new or first-time practitioners. Generally, practitioners take the neurotoxin in solid powder form and inject a certain volume of saline solution into the neurotoxin vial to prepare a deliverable neurotoxin with a desired concentration. Once the saline solution has been mixed with the neurotoxin, another syringe with a relatively long needle is used to draw up the reconstituted neurotoxin into the barrel of the syringe. After the syringe has been charged with a certain volume of neurotoxin, the long needles are switched for shorter injection needles. This switching often results in wasting valuable neurotoxin product that is left behind in the long needles or that leaks during the switch.

Conventionally, practitioners have tried to solve this problem by starting with the short injection needles already attached to the syringe barrel and simply inserting the needle through the rubber stop lid and withdrawing the reconstituted neurotoxin. However, not only does this practice dull the injection needle (thus making subsequent delivery injections more painful to the user), but the short injection needles are generally not long enough to reach the bottom of the neurotoxin vial, and are thus unable to withdraw all of the reconstituted neurotoxin. Further, if a short needle is used to withdraw toxin and the vial is inverted in order to withdraw the dregs, the last drops of liquid settle to the side of the rubber stopper where it is inaccessible by the needle. Thus, practitioners have tried to use longer injection needles and manually hold the neurotoxin vial at an angle in order to move the reconstituted neurotoxin dregs onto one side of the bottom surface in order to extract as much neurotoxin as possible. Such a maneuver, however, is generally unstable as practitioners have to use both hands to maneuver the syringe and the vial into a proper orientation in order to withdraw the neurotoxin dregs from the vial. Further, even the use of these longer needles results in excess neurotoxin product being left behind in the long needles.

Practitioners are often frustrated and perplexed with the shortcomings of neurotoxin preparation and injection procedures. Accordingly, practitioners tend to modify the concentration of the reconstitute neurotoxin to avoid wasting product. For example, a practitioner may inject less saline into the vial during reconstitution, thus making the neurotoxin more concentrated and thus requiring fewer instances of drawing up neurotoxin into the injection syringes. However, the wasted value of any product that remains in the needle or in the vial is greater than before because the medication has a higher concentration. Practitioners have also tried to solve these problems by using more saline to over-dilute the reconstituted neurotoxin. In such a procedure, while any neurotoxin left behind in the vial or in the needle is not as wasteful because the product is less concentrated, a practitioner would still have to redraw diluted neurotoxin into his injection syringe more frequently in order to administer the proper quantity of neurotoxin. The practice of repeatedly redrawing more neurotoxin not only takes more of the practitioner and patient's time, but the repeated refilling and injecting may result in other possible complications, such as inaccurate dosage administration, contamination, needle dulling, and neurotoxin leakage, among other complications. Therefore, conventional methods, systems, and procedures for preparing and injecting neurotoxins are difficult, perplexing, time consuming, and costly for many practitioners.

### BRIEF DESCRIPTION OF THE DRAWINGS

In order that the advantages of the subject matter of the present disclosure will be readily understood, a more particular description of the subject matter will be rendered by reference to specific embodiments that are illustrated in the appended drawings. Understanding that these drawings depict only typical embodiments of the subject matter of the present disclosure and are not therefore to be considered to be limiting of its scope, the subject matter will be described and explained with additional specificity and detail through the use of the accompanying drawings, in which:

FIG. 1 is a perspective view of one embodiment of a portable neurotoxin injection tray;

FIG. 2 is a perspective view of another embodiment of a portable neurotoxin injection tray;

FIG. 3 is a top view of one embodiment of a portable neurotoxin injection tray;

FIG. 4A is a cross-sectional side view of one embodiment of a portable neurotoxin injection tray;

FIG. 4B is a cross-sectional side view of a neurotoxin vial depicting a tilted orientation, according to one embodiment;

FIG. 5A is a side view of one embodiment of an injection syringe and a neurotoxin vial;

FIG. 5B is a side view of one embodiment of an injection syringe that has a syringe barrel sleeve and a neurotoxin vial;

FIG. 5C is a perspective view of one embodiment of a syringe cap and a syringe cap holder;

FIG. 5D is a side view of one embodiment of an injection syringe, a dosage alignment cap with a needle channel, a neurotoxin vial, and a siphoning needle;

FIG. 5E is a side view of one embodiment of an injection syringe, a dosage alignment cap with a syringe barrel channel, and a neurotoxin vial;

FIG. 6A depicts one embodiment of a vial opener device;

FIG. 6B depicts another embodiment of a vial opener device; and

FIG. 7 is a schematic flow chart diagram of a method for using a portable neurotoxin injection tray, according to one embodiment.

### SUMMARY

The subject matter of the present disclosure has been developed in response to the present state of the art. Accordingly, the subject matter of the present disclosure has been developed to provide an apparatus, system, and method for safely and efficiently administering neurotoxin injections that overcomes many or all or some shortcomings in the prior art.

According to one embodiment, a portable neurotoxin injection tray apparatus includes a vial section with multiple compartments. Each compartment is capable of storing at least one neurotoxin vial or at least one saline vial. The apparatus also includes a dosage compartment with a tilted support member for positioning at least one of the neurotoxin vials in a tilted orientation. Further, the apparatus includes an injection syringe section that includes multiple compartments, where each compartment is designated to store at least one injection syringe filled with a different dosage of neurotoxin.

In some implementations of apparatus, the dosage compartment further includes at least one viewing window that allows a user to see the at least one neurotoxin vial in the tilted orientation. According to certain implementations, the apparatus may further include a syringe cap holder, a vial opener device compartment, sterile pads compartment, and/or a medical waste disposal compartment. The medical waste disposal compartment can be detachable from the apparatus. In some implementations, the apparatus can also include a cooling pack chamber, a saline syringe section capable of storing at least one saline syringe, a siphoning needle compartment, and/or a dosage alignment cap compartment. The saline syringe may be used to transfer saline solution from the at least one saline vial to the at least one neurotoxin vial.

In another embodiment, a method for using a portable neurotoxin injection tray apparatus includes providing an injection tray apparatus. The injection tray apparatus includes a vial section with multiple compartments, where each compartment is holding at least one neurotoxin vial or at least one saline vial. The apparatus also includes a saline syringe section that holds at least one saline syringe, a dosage compartment that includes a support member and a neurotoxin vial positioned on the support member and situated in a tilted orientation, a vial opener device compartment, and an injection syringe section that includes multiple injection syringe compartments. Each injection syringe compartment holds injection syringes containing a specific dosage of neurotoxin. The method further includes removing a cap from the neurotoxin vial in the dosage compartment using a vial opener device stored in the vial opener device compartment, reconstituting the neurotoxin vial in the dosage compartment by injecting saline solution from one of the at least one saline vial into the neurotoxin vial, and preparing the injection syringes by filling injection syringes with different specific dosages of neurotoxin from the reconstituted neurotoxin vial.

According to some implementations, the method includes injecting patients with a desired quantity of neurotoxin by using the injection syringes with the different specific dosages from the injection syringe section. Injecting patients may include selecting multiple injection syringes from the multiple injection syringe compartments, wherein the selected multiple injection syringes includes at least one injection syringe from at least two multiple injection syringe compartments. In some implementations, before preparing the injection syringes by filling the injection syringes, the method includes placing a dosage alignment cap on the neurotoxin vial.

In yet another embodiment, a siphoning apparatus includes a dosage alignment cap coupled to a vial. The dosage alignment cap includes a needle channel in an angled orientation with respect to a central axis of the vial. The apparatus also includes a siphoning needle insertable within the needle channel. The siphoning needle is capable of extending to the bottom edge of the vial for extracting dregs, and the siphoning needle is attachable to an injection syringe. The apparatus may further include a supporting feature for maintaining the siphoning needle in the angled orientation.

Reference throughout this specification to features, advantages, or similar language does not imply that all of the features and advantages that may be realized with the subject matter of the present disclosure should be or are in any single embodiment of the subject matter. Rather, language referring to the features and advantages is understood to mean that a specific feature, advantage, or characteristic described in connection with an embodiment is included in at least one embodiment of the subject matter of the present disclosure. Thus, discussion of the features and advantages, and similar language, throughout this specification may, but do not necessarily, refer to the same embodiment.

Furthermore, the described features, structures, advantages, and/or characteristics of the subject matter of the present disclosure may be combined in any suitable manner in one or more embodiments and/or implementations. In the following description, numerous specific details are provided to impart a thorough understanding of embodiments of the subject matter of the present disclosure. One skilled in the relevant art will recognize that the subject matter of the present disclosure may be practiced without one or more of the specific features, details, components, materials, and/or methods of a particular embodiment or implementation. In other instances, additional features and advantages may be recognized in certain embodiments and/or implementations that may not be present in all embodiments or implementations. Further, in some instances, well-known structures, materials, or operations are not shown or described in detail to avoid obscuring aspects of the subject matter of the present disclosure. The features and advantages of the subject matter of the present disclosure will become more fully apparent from the following description and appended claims, or may be learned by the practice of the subject matter as set forth hereinafter.

### DETAILED DESCRIPTION

Similarly, reference throughout this specification to “one embodiment,” “an embodiment,” or similar language means that a particular feature, structure, or characteristic described in connection with the embodiment is included in at least one embodiment of the subject matter of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the subject matter of the present disclosure, however, absent an express correlation to indicate otherwise, an implementation may be associated with one or more embodiments.

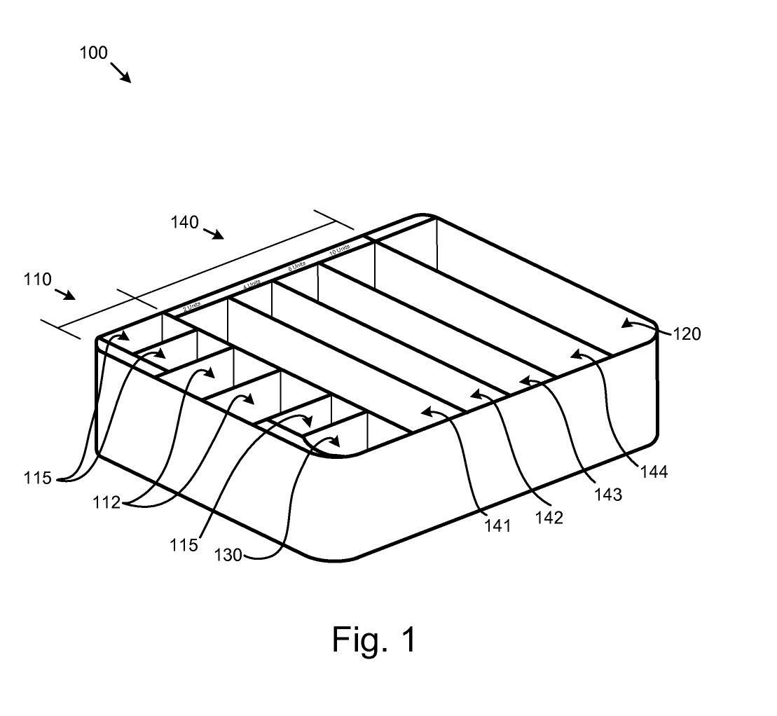

FIG. 1 is a perspective view of one embodiment of a portable neurotoxin injection tray 100. The portable neurotoxin injection tray 100 includes a vial section 110, a saline syringe section 120, a dosage compartment 130, and an injection syringe section 140. The injection tray 100 is an apparatus that can be used by neurotoxin practitioners to prepare, store, and administer neurotoxin injections. The injection tray 100 has a structure that includes various sections and compartments for organizing the materials and components in order to facilitate a practitioner's successful and efficient administration of a medical or cosmetic neurotoxin treatment. The tray 100 may be constructed from any material that is suitable for portably holding and storing medical supplies, such as plastic, stainless steel, or composite materials, among others. The tray 100 may be a single integrated material or may include moveable and or slidable partitions that form each of the compartments that are discussed below. In other words, the tray 100 may include various components, sections, and compartments that are modular in fashion and various other components, sections, and compartments that are integrated together. Additionally, the positioning, configuration, arrangement, and orientation of the various sections and compartments 110, 120, 130, 140 may differ from the embodiment depicted in FIG. 1. In other words, the scope of the present disclosure is not limited to the specific layout of the compartments as depicted in the accompanying figures. Also, the tray may include elements that facilitate the portability of the tray. For example, handles, straps, mounting elements, and moveable platforms may be implemented with the present disclosure.

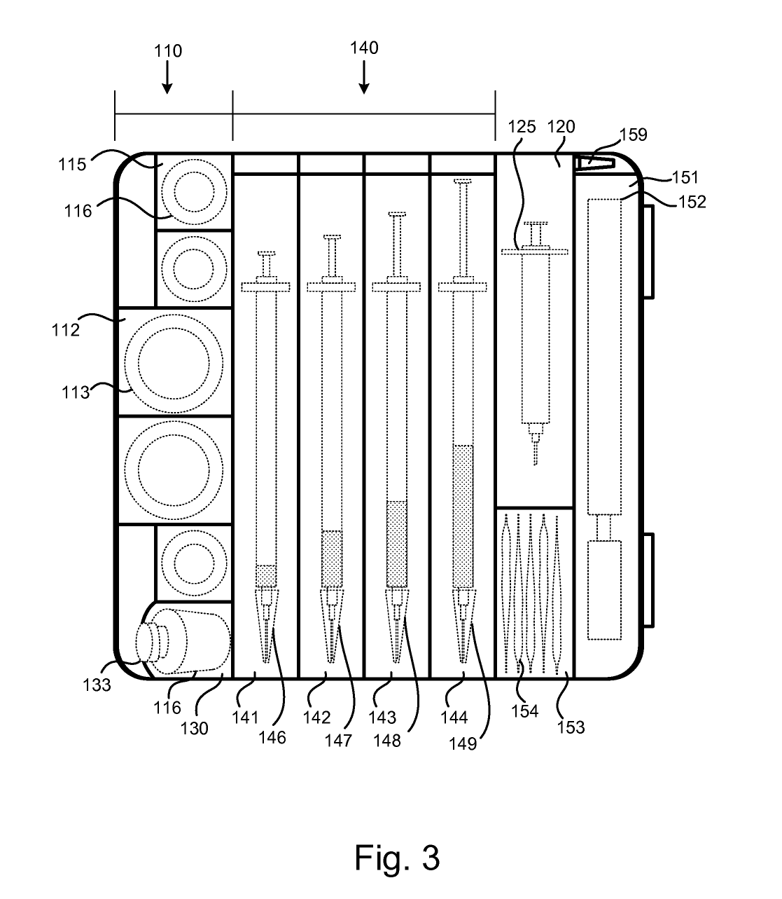

As depicted and according to one embodiment, the injection tray 100 may include a vial section 110 with multiple sub-sections or compartments. These compartments may include saline vial compartments 112 and neurotoxin vial compartments 115. As indicated by the respective names, the saline vial compartments 112 are for holding saline vials and the neurotoxin vial compartments 115 are for holding neurotoxin vials. Although it is contemplated that a single saline vial compartment 112 and/or a single neurotoxin vial compartment 115 may be used in the injection tray 100, the plural word ‘compartments’ will be used throughout the present disclosure. In other words, the phrase ‘saline vial compartments’, for example, is not intended to restrict the scope of the disclosure to trays that have at least two compartments for holding saline vials. In the depicted embodiment, the tray 100 includes two saline vial compartments 112 that are each slightly larger than the four neurotoxin vial compartments 115. The number of vial compartments in the vial section 110 is not limited to the number depicted in FIG. 1 and it is contemplated that more of each type or fewer of each type may be implemented in a certain embodiment of an injection tray, according to the specifics of a given application. Additional details regarding the vials and their contents are included below with reference to FIG. 3.

The injection tray 100 also includes a saline syringe section 120. The saline syringe section 120, according to one embodiment, may include multiple compartments or may be one single compartment (as depicted). The saline syringe section 120 holds the saline supply syringes that are used to withdraw saline solution from the saline vials in the saline vial compartments 112 and inject the saline solution into the neurotoxin vials 115 for reconstitution. Additional details regarding the saline supply syringes are included below with reference to FIG. 3.

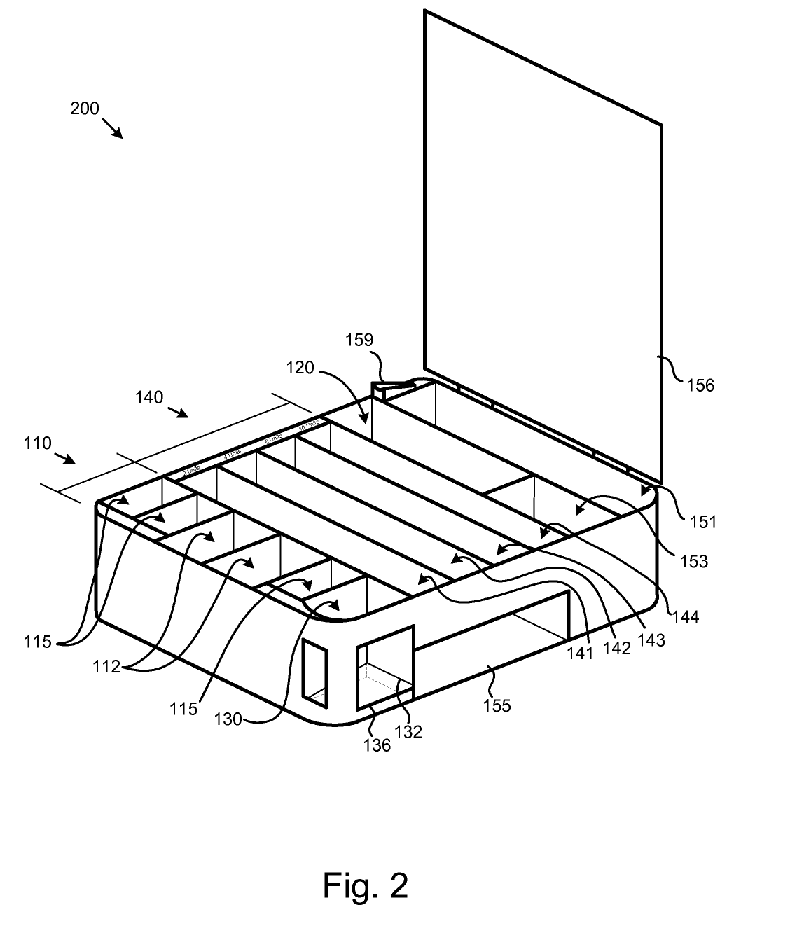

The injection tray 100 further includes a dosage compartment 130. The dosage compartment 130 is a location in the tray 100 where a reconstituted neurotoxin vial is placed while injection syringes withdraw specific dosages of neurotoxin from the vial. The dosage compartment 130 includes a support member that configures the neurotoxin vial in a tilted orientation, thus facilitating a practitioner's ability to withdraw the neurotoxin dregs from the vial. Additional details regarding the dosage compartment 130 are included below with reference to FIGS. 2 and 3.

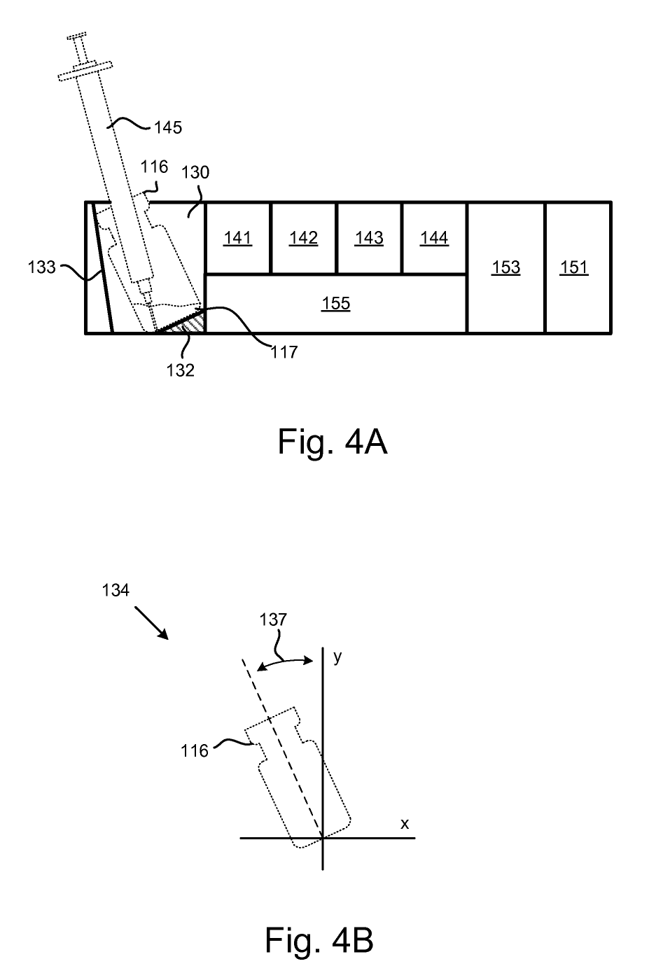

The depicted injection tray 100 also includes an injection syringe section 140 that has multiple subsections or compartments. The injection syringe compartments 141-144 are each capable of holding at least one injection syringe filled with a specific dosage of neurotoxin. For example, the first injection dosage compartment 141 may hold injection syringes that have each been filled with 2 units of neurotoxin while the second injection dosage compartment 142 may hold injection syringes that have each been filled with 4 units of neurotoxin, etc. FIG. 2 depicts four separate injection dosage compartments, but it is contemplated that there may be more or fewer dosage compartments according to a specific application. In one embodiment, the injection syringe section 140, or at least a few of the injection syringe compartments, may be individually portable and removable. For example, in one embodiment there may be multiple injection syringe compartments that can be switched out as injections syringes are used. In such an embodiment, a practitioner may fill extra injection syringes with specific neurotoxin dosages and store the extra injection syringes in removable injection syringe compartments. As needed, the practitioner may then replace his supply of injection syringes in the tray apparatus by removing an empty compartment and replacing it with an extra injection syringe compartment that contains prepared injection syringes. These portable/extra injection syringe compartments may also have their own lid so as to prevent the prepared injection syringes from falling out of the compartment. Additional details regarding the syringe section 140 are included below with reference to FIG. 4A.

FIG. 2 is a perspective view of another embodiment of a portable neurotoxin injection tray 200. In the depicted embodiment, the tray 200 includes the sections and compartments described above with reference to FIG. 1, specifically a vial section 110, a saline syringe compartment 120, a dosage compartment 130, and an injection syringe section 140. However, FIG. 2 further includes a vial opener compartment 151, a sterile pads compartment 153, a cooling pack chamber 155, a hinged lid 156, and a syringe cap holder 159. The positioning, configuration, arrangement, and orientation of the various sections and compartments 110, 120, 130, 140, 151, 153, 155, 156, 159 may vary from the embodiment depicted in FIG. 2. In other words, the scope of the present disclosure is not limited to the specific layout of the compartments as depicted in the accompanying figures. Additionally, it is contemplated that an injection tray of the present disclosure may only have a few of the various components, sections, and compartments or may have additional compartments not discussed in detail in the pages of this disclosure. For example, although not depicted in the figures, it is contemplated that the injection tray may also include a medical waste compartment, a sharps disposal compartment, a first aid compartment, a water soluble skin-marking pen compartment, etc.

The tray, according to one embodiment, may include a dosage compartment 130 that has a viewing window 136 built into one or several of the exterior walls that surround the compartment. Through the viewing window 136, a practitioner can see a neurotoxin vial in a tilted orientation in order to ascertain the remaining contents of the neurotoxin vial. In one embodiment, the viewing window 136 may be a glass or a transparent composite/plastic material. In another embodiment, the viewing window 136 may be an open hole in the wall of the tray 200. The support member 132 depicted in FIG. 2 is a ramp that can be seen through the viewing window 136. The ramp 132 elevates one side of the neurotoxin vial so that the dregs collect on the other side. With the neurotoxin vial configured in a tilted orientation in front of the viewing window 136, a practitioner can monitor the level of neurotoxin remaining in the vial and can withdraw the neurotoxin dregs from the vial. Additional details relating to the support member 132 and the dosage compartment are included below with reference to FIG. 4.

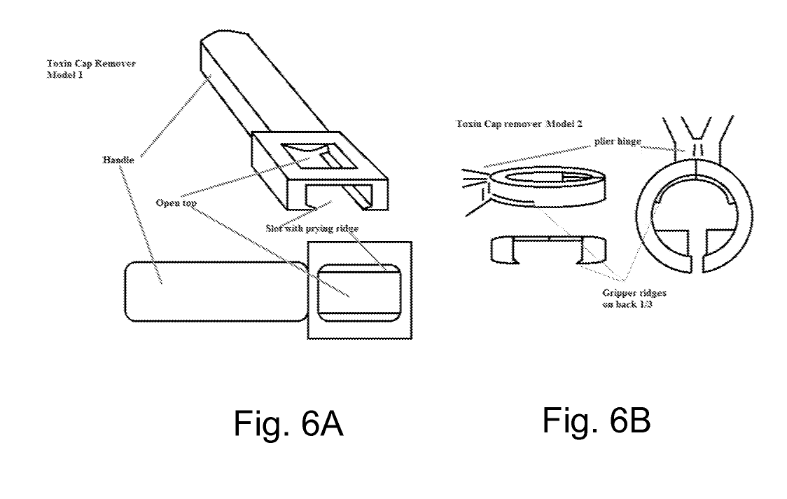

The vial opener compartment 151 holds a vial opener device. A practitioner can use the vial opener device to remove the neurotoxin vial caps. As described in greater detail below, in one embodiment of the present disclosure, the barrels of the injection syringes used are thin enough to insert into the mouth of the neurotoxin vial in order to withdraw the reconstituted neurotoxin. Thus, the vial opener device is used to remove the cap from the vial in order to make room for the injection syringe barrels to fit within the mouth of the vial. FIGS. 6A and 6B include details regarding several embodiments for vial opener devices that can be used in the present disclosure.

The sterile pads compartment 153 may hold several sterile pads or other sanitizing agents for sterilizing the various components and/or the injection site on the patient. The sterile pads compartment 153, according to one embodiment, may include multiple, individually wrapped sterile sheets. In another embodiment, the sterile pads compartment 153 may include antiseptic solutions for cleaning an area on the patient's body or local anesthetic compounds for numbing the area on the patient's body where the injection will occur. Further, the sterile pads compartment 153 may also hold clean gauze sponges.

The tray 200 depicted in FIG. 2 also includes a cooling chamber 155. Certain neurotoxins, once reconstituted, must be maintained below some maximum temperature to prevent degradation of the product. The maximum temperature often is below room temperature. In one embodiment, the cooling chamber 155 is centrally located in the tray 200 so as to cool the contents of the tray. In one embodiment, the cooling pack may be removable from the chamber 155 and may be refrigerated independent of the tray 200. In another embodiment, the cooling pack is sealed into or integrally formed within the chamber 155, thus the entire tray 200 may need to be placed into a refrigerator periodically to maintain the temperature of the cooling pack. In one embodiment, the cooling chamber 153 may not actually hold a cooling pack but may include a cooling system. For example, the cooling chamber 153 may include an independent, battery operated coolant system so that a practitioner can transport the neurotoxin to remote locations or can administer injections for a longer time period than would otherwise be allowable if cooling packs were used. In another embodiment, the cooling chamber 153 may be a solid-state refrigeration unit that includes a power cord for connecting the unit to an electrical outlet. The power cord may be detachable from the cooling chamber 153 to allow a practitioner to move about a room administering neurotoxin treatments. Also, in another embodiment the cooling chamber 155 may extend across the entire bottom of the tray 200. In yet another embodiment, the cooling chamber 155 may also be located in the lid 156 of the tray 200. In yet another embodiment, the tray apparatus may be placed into a portable cooling device. For example, the tray 200 may be placed into a cooler during transportation to an administration site or between patient injections.

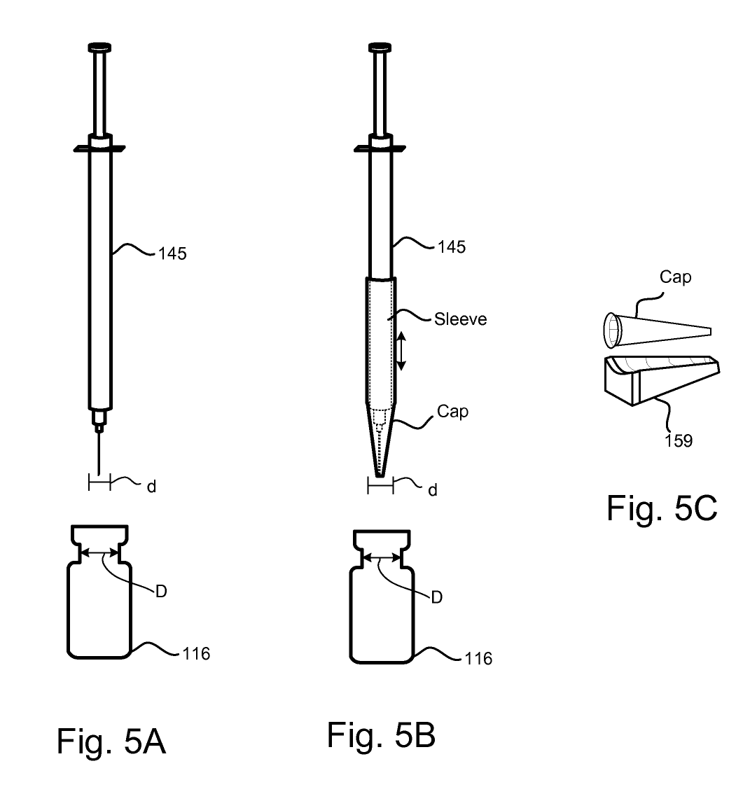

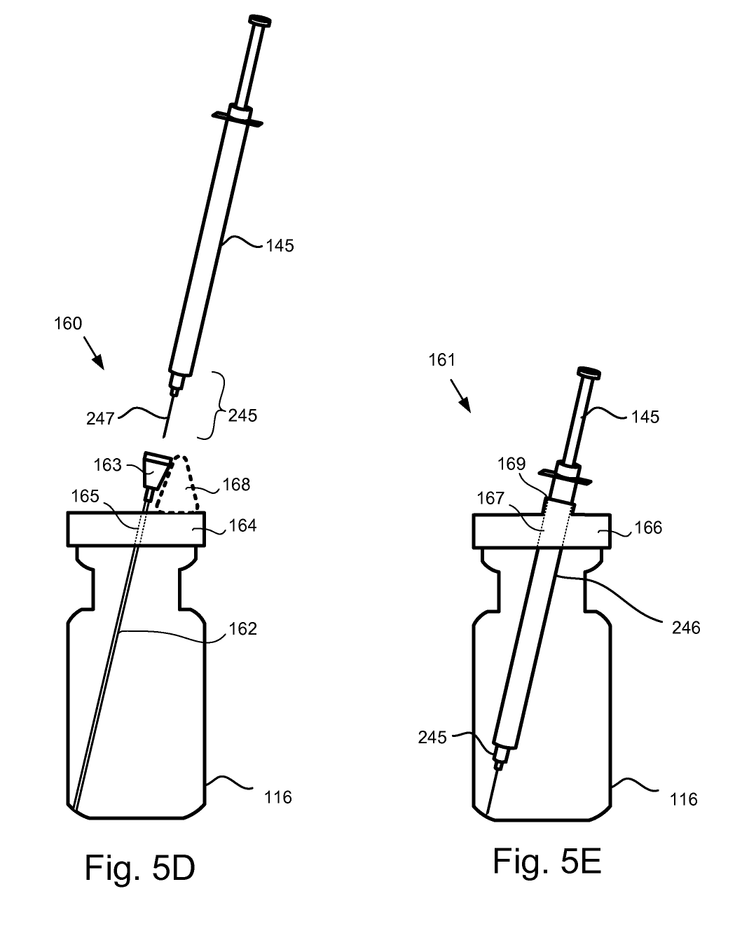

As depicted, the tray apparatus 200 may also include a lid 156 that can close over the top of the tray 200. In one embodiment, the lid 156 may be coupled to the tray 200 via hinges so that the lid 156 can rotate over the top of the tray 200 and enclose the contents, thus preventing damage to the contained components and preventing potential spills while transporting the tray from location to location. In another embodiment, the lid 156 may be an independent component and may not be coupled to the tray 200. Further, the lid 156 may also include a fastening mechanism that interacts with a coordinating fastening mechanism on the tray 200 so as to securely close the lid 156 over top of the tray 200. The neurotoxin injection tray 200 may also include a syringe cap holder 159. The syringe cap holder 159 may be used help a user remove and replace the cap to the syringe needle during the preparation and injection of the neurotoxin. Additional details regarding the syringe cap holder 159 are included below with reference to FIG. 5C.

FIG. 3 is a top view of the portable neurotoxin injection tray of FIG. 2, according to one embodiment. In the depicted embodiment, the tray includes the sections and compartments described above with reference to FIGS. 1 and 2, specifically a vial section 110, a saline syringe compartment 120, a dosage compartment 130, an injection syringe section 140, a vial opener compartment 151, a sterile pads compartment 153, a cooling chamber 155, a hinged lid 156, and a syringe cap holder 159. The positioning, configuration, arrangement, and orientation of the various sections and compartments 110, 120, 130, 140, 151, 153, 155, 156, 159 may vary from the embodiment depicted in FIG. 3. In other words, the scope of the present disclosure is not limited to the specific layout of the compartments as depicted in the accompanying figures.

The vial section 110 includes saline vial compartments 112 for holding saline vials 113 and neurotoxin vial compartments 115 for holding neurotoxin vials 116. In the depicted embodiment, the saline vials 113 are comparatively larger than the neurotoxin vials 116. The saline vials 113, according to one embodiment, contain a saline solution that does not have any preservatives. The neurotoxin vials 116 contain a neurotoxin, either in a pre-application state (i.e., dry solid powder) or in a reconstituted state. In one implementation, the neurotoxin vials contain 100 units of type A botulinum toxin, as manufactured and produced by BOTOX cosmetic of Allergan Inc. in Irvine, Calif., USA.

The dosage compartment 130, as described briefly above, is configured to hold a neurotoxin vial 116 in a tilted configuration. The dosage compartment 130 may have a tilted or a curved wall 133 or may have multiple tilted or curved walls that facilitate the stability of the neurotoxin vial 116 while in the tilted orientation. As depicted in FIG. 3, the dosage compartment 130 may be one of the compartments in the vial section 110 of the tray. The saline syringe section 120 may include one or multiple saline supply syringes 125 for transferring the proper amount of saline solution from the saline vials 113 into the neurotoxin vials 116. The saline syringes 125 may be the same as the injection syringes (described below) or the saline syringes 125 may be different. For example, the saline syringes 125 may have a higher volume capacity than the injection syringes, thus allowing for the proper volume of saline solution to be quickly and easily transferred to the neurotoxin vials 116.

The injection syringes 146-149, according to one embodiment, are hubless insulin syringe-type syringes. These syringes may have the volume markings along the barrel replaced with specific dosage or unit markings corresponding to a specific dosage of neurotoxin contained therein. The dosage compartments 141-144 may also have labels signifying how many units of neurotoxin are within each injection syringe contained in that compartment. For example, the injection syringes 146 in the first injection dosage compartment 141 may all be filled with a specific dosage of neurotoxin, say 2 units. The injection syringes 147 in the second injection dosage compartment 142 may all be filled with another dosage of neurotoxin, say 4 units. Thus, each dosage compartment may hold syringes containing the same units of neurotoxin. Additional details regarding the injection syringes and the process for filling them are included below with reference to FIGS. 4A, 5A-5C and 7. FIG. 3 also depicts the sterile pads compartment 153 holding multiple sterile pads 154, a vial opener compartment 151 holding a vial opener device 152 (described below with reference to FIGS. 6A and 6B), and a syringe cap holder 159 (described below with reference to FIG. 5C).

FIG. 4A is a cross-sectional side view of one embodiment of a portable neurotoxin injection tray with an injection syringe 145 inserted into a neurotoxin vial 116 that is in a tilted orientation in the dosage compartment 130. According to one embodiment, the dosage compartment 130 includes a support member 132 to orient the neurotoxin vial 116 in the tilted orientation. In one embodiment, the support member 132 may be integrated into the bottom of the dosage compartment 130. In another embodiment, the support member 132 may be removable and may be placed into the bottom of the dosage compartment 130 in a variety of directions so that a practitioner may orient the tilt of the vial 116 in a direction most suited to his preferences. In one embodiment, the support member 132 comprises a wedge like ramp, as depicted. In another embodiment, the support member 132 may comprise a plate, a pole, a shaft or other element configured to tilt the vial. With the vial 116 in the tilted orientation, an injection syringe 145 may be inserted into the vial (with the vial cap removed) in order to withdraw the dregs of the neurotoxin medication 117.

FIG. 4B is a cross-sectional side view of a neurotoxin vial 116 depicting a tilted orientation 134, according to one embodiment. As described above, the dosage compartment has a support member 132 that tilts the vial 116 at a certain angle 137. Depending on the support member 132 and the configuration and dimensions of the dosage compartment 130, the angle 137 may be selected according to the specifics of a given application or according to the specific dimensions of the neurotoxin vial 116.

FIG. 5A is a side view of one embodiment of an injection syringe 145 and a neurotoxin vial 116. The injection syringes 145 used in the present disclosure, according to one embodiment, are insulin-type syringes that have a barrel narrow enough (d) to fit within the mouth (D) of a neurotoxin vial 116. As depicted in FIG. 4A, this allows a practitioner to insert the injection syringe 145, even a portion of the barrel of the syringe, into the vial to withdraw neurotoxin medication 117. Because the barrel is able to fit within the vial 116 (even when the barrel is angled with respect to the vertical/longitudinal axis of vial 116), the practitioner is able to use syringes that have comparatively shorter needles and thus the amount of neurotoxin medication left behind in the needle is decreased.

FIG. 5B is a side view of one embodiment of an injection syringe that has a syringe barrel sleeve and a neurotoxin vial. Since the barrels of the syringes 145 in the present disclosure are being inserted into the vials 116 in at least one embodiment, there is a possibility for contamination, or at least a possibility for the perception of contamination. Thus, according to one embodiment, syringe sleeves may be implemented to cover the barrel of the syringes 145 to maintain the sterile nature of the injection procedure. The sleeves may be slidably attached to the syringe barrel 145 and may be removable and disposable. In another embodiment, the sleeves may be attached to or integrally formed with the needle cap and thus may be removed and/or replaced at the same time as the needle cap.

FIG. 5C is a perspective view of one embodiment of a syringe cap and a syringe cap holder 159. The syringe cap holder 159 may be positioned on the injection tray and may be configured to facilitate the practitioner with removing, holding, and/or replacing the needle cap. In one embodiment, the syringe cap holder 159 is merely a depression in a surface of the tray where a practitioner may place removed syringe caps while filling the syringe or using the syringe to inject neurotoxin. For example, a practitioner may manually remove a syringe cap and place it into a slot (the syringe cap holder 159) on the tray to prevent the cap from falling to the floor or from otherwise getting lost or contaminated. The syringe cap holder 159 may be a passive component that provides enough resistance so that a practitioner can re-insert a needle into a cap that is resting in the cap holder 159 and lift the cap up and out of the cap holder 159 to manually fasten/tighten the cap on the syringe.

In another embodiment, as depicted, the syringe cap holder 159 may have a curved surface that contours the curved surface of a needle cap. Further, the syringe cap holder may have a groove on a proximal side corresponding to a lip on the cap. With such a configuration, the practitioner may take an injection syringe 145, with a cap in place over the needle, and may position the cap into the curved surface of the holder 159. When the lip on the cap aligns with the groove in the holder 159, the practitioner may pull back on the syringe and the cap will remain in place while the practitioner fills the syringe or injects the medication. Once finished with the syringe, the practitioner can once again recap the syringe needle by inserting the needle into the cap and snapping the cap into place before lifting the cap from the cap holder 159.

FIG. 5D is a side view of one embodiment of a siphoning apparatus 160. The siphoning apparatus 160 includes a dosage alignment cap 164 with a needle channel 165 and a siphoning needle 162. In one embodiment, the dosage alignment cap 164 may be a conventional cap (e.g., a non-permeable membrane) that is pierced by the siphoning needle 162. For example, a conventional rubber stopper of a vial may be modified to have a needle channel 165 that receives and orients the siphoning needle 162. For example, the siphoning apparatus 160 may be implemented using conventional caps/stoppers that are retrofitted to include a needle channel 165.

In another embodiment, the dosage alignment cap 164 may replace the original/conventional cap on the neurotoxin vial 116. The dosage alignment cap 164 has a needle channel 165 through which a siphoning needle 162 is inserted. Thus, the siphoning needle 162 may be an attachment that is received into the needle channel 165. In another embodiment, the siphoning needle 162 is an integrated component of the dosage alignment cap 164. In other words, the needle channel 165 may refer to a portion of the siphoning needle 162 that is integrated (e.g., unitary with) the dosage alignment cap 164.

The needle channel 165 may be oriented so that the siphoning needle 162 is directed towards the bottom edge of the neurotoxin vial 116 to facilitate the extraction of all of the neurotoxin. As described above with reference to FIGS. 4A and 4B, the neurotoxin vial 116 may be supported in an angled orientation, thus allowing the dregs to settle into the bottom corner/side of the neurotoxin vial 116. Accordingly, the dosage alignment cap 164 may be configured so that the needle channel 165 is angled relative to a central axis of the vial to direct the distal end of the siphoning needle attachment 162 to the bottom corner/side of the neurotoxin vial 116. Further, the distal end of the siphoning needle attachment 162 may be tapered and configured to abut the rounded bottom edge of the neurotoxin vial 116, thus further promoting the extraction of the last dregs of neurotoxin from the neurotoxin vial 116. The dosage alignment cap 164 may be constructed of various materials, such as rubber, plastic, glass, metal, etc.

Injection syringes 145 can be coupled to the siphoning needle 162 via a connector 163. The connector 163 may couple to the needle-end 245 of an injection syringe 145 via a threaded engagement, a resistive fit, or a Luer-lock fitting, among others. The needle 247 of the injection syringe 145 is inserted within the siphoning needle 162 of the siphoning apparatus 160 (i.e., concentric needles). In other words, the injection syringe 145, which already has a needle 247, is inserted within and coupled to the siphoning needle 162 to form a needle-over-needle or needle-within-needle configuration. Accordingly, the engagement between the outer surface of the injection needle 247 and the inner surface of the siphoning needle 162 may be such that minimal or no void space is between the two concentric needles 162, 247, thus preventing loss of neurotoxins (e.g., by prohibiting neurotoxins from being trapped between the two needles 162, 247), and facilitating the siphoning/suctioning engagement between the two needles 162, 247.

As described above in the Background section, conventional systems and procedures generally involve attaching a relatively long and larger “drawing” needle and associated hub to the syringe in order to draw up the reconstituted neurotoxin into the barrel of the syringe before replacing the “drawing” needle with an injection needle and associated hub. This switching often results in wasting valuable neurotoxin product that is left behind in the needle shaft and/or the needle hub. Practitioners have tried to solve this problem by starting with the short injection needle already attached to the syringe barrel and simply inserting the injection needle through the rubber stop lid and withdrawing the reconstituted neurotoxin. However, not only does this practice dull the injection needle (thus making subsequent delivery injections more painful to the user), but the short injection needles are generally not long enough to reach the bottom of the neurotoxin vial, and are thus unable to withdraw all of the reconstituted neurotoxin.

Accordingly, the siphoning apparatus 160 of the present disclosure implements the needle-over-needle configuration (i.e., the needle 247 of the injection syringe 145 is inserted within the siphoning needle 162) to prevent wasting the neurotoxin and prevent dulling the injection needle 247. In other words, because the siphoning needle 162 is a larger needle and provides the penetration into and through the rubber stop lid, the needle 247 does not need to be replaced and does not penetrate the rubber stop lid. In such an embodiment, hubless injection syringes 145 may be employed because there is no need for the injection syringes 145 to have swappable or hubbed needles (e.g., charging/drawing and injection type needles).

As depicted in FIG. 5D, a supporting feature 168 may also be included to support and secure the siphoning needle 162 in a desired position. For example, for some embodiments, an angled needle channel 165 may not be pre-formed in the dosage alignment cap. Accordingly, a supporting feature 168 may be used to maintain the siphoning needle 162 in a certain angled orientation through a conventional cap. The supporting feature 168 may be any of various structure capable of maintaining in place the siphoning apparatus 160 in a desired position, such as, for example, a wedge, tab, notch, tie, strap, pad, or fastener, among others. In some implementations, the supporting feature 168 is removably coupled to the vial 116 and/or the dosage alignment cap 164. In another embodiment, the siphoning apparatus 160 may be used to fill several injection syringes 145 and may also be subsequently used on different neurotoxin vials 116.

Although not depicted, the tray apparatus may further include a section or a compartment for holding one or more dosage alignment caps 164, and the tray may include a section or compartment for holding one or more siphoning needles 162. In another embodiment, the neurotoxin vials 116 may be pre-fabricated to include the angled needle channel 165, thus eliminating the need to remove and replace an existing cap. In such embodiments, it may be necessary to initially remove a seal or membrane that isolates the contained neurotoxin from the atmosphere during storage/transportation. For example, a removable aluminum tab could cover the exterior aperture of the needle channel 165. Alternatively, the cap may include a scored pattern or a “near-perforated” pattern stamped in the cap that would operably rupture along the scored/perforated patterns when inserting the siphoning needle 162. In another embodiment, the connector end 163 of the siphoning needle 162 may have a membrane, constructed from rubber, latex, or other similar material, that covers the siphoning channel 165 and substantially isolates the neurotoxin in the vial from the atmosphere. In such an embodiment, the needle 247 of the injection syringes 145 would pierce the membrane in order to draw neurotoxin into the injection syringe 145 via the siphoning needle 162.

FIG. 5E is a side view of another embodiment of a siphoning apparatus 161. The siphoning apparatus 161 includes a dosage alignment cap 166 with a syringe barrel channel 167. In one embodiment, as described above, the dosage alignment cap 166 can replace the original/conventional cap on the neurotoxin vial 116. The dosage alignment cap 166, according to one implementation, has a syringe barrel channel 167 through which the barrel 246 of the injection syringe 145 can be inserted. The syringe barrel channel 167 may be angled with respect to a central axis so that the injection syringe 145 is directed towards the bottom edge of the neurotoxin vial 116 to facilitate the extraction of all of the neurotoxin. As described above with reference to FIGS. 4A and 4B, the neurotoxin vial 116 may actually be supported in an angled orientation itself, thus allowing the dregs to settle into the bottom corner/side of the neurotoxin vial 116. The dosage alignment cap 166 may be configured so that the syringe barrel channel 167 directs the distal end 245 of the injection syringe 145 to the bottom corner/side of the neurotoxin vial 116.

The dosage alignment cap 166 may be constructed of various materials, such as rubber, plastic, glass, metal, etc. As depicted in FIG. 5E, the dosage alignment cap 166 may include, or be implemented with, a supporting feature 169 that supports and secures the injection syringe 145 in a desired position. For example, depending on the width of the dosage alignment cap 166, the supporting feature 169 may actually be a taller or thicker section of the cap 166, thus making the syringe barrel channel 167 longer to improve the lateral support of an inserted injection syringe 145 in the proper orientation for extracting the dregs from the neurotoxin vial 116.

As described above with reference to the siphoning apparatus 160 of FIG. 5D, the dosage alignment cap 166 of the siphoning apparatus 161 in FIG. 5E may be a conventional cap (e.g., a non-permeable membrane) that has been modified to include a syringe barrel channel 167. For example, a conventional rubber stopper of a vial may be modified to have a bore hole that functions as the syringe barrel channel 167 to receive and orients the syringe barrel 246. In other words, the siphoning apparatus 161 may be implemented using conventional caps/stoppers that are retrofitted to include a syringe barrel channel 167. In another embodiment, the dosage alignment cap 166 may replace the original/conventional cap on the neurotoxin vial 116.

FIG. 6A depicts one embodiment of a vial opener device. The vial opener device, as briefly described above, may be a hinged or plier-like device that has gripping teeth or a gripping ridge on the posterior inferior of the device to allow a practitioner to leverage the removal of the aluminum cap from a neurotoxin vial. In one embodiment, the vial opener device may also be used to remove the cap from a saline vial. In another embodiment, the caps on the saline vials do not need to be removed. In other words, the saline supply syringes may simply withdraw saline in a conventional manner by inserting the saline supply syringe needle through the rubber stopper of the saline vials.

FIG. 6B depicts another embodiment of a vial opener device. In another embodiment, the vial opener device is a slide-on type device that has lateral edges that slide underneath the lateral inferior edges of the aluminum rim of the vial cap. Once again, the practitioner may leverage the device to pry off the vial cap. The tray of the present disclosure may include a vial opener compartment where the vial opener device can be held.

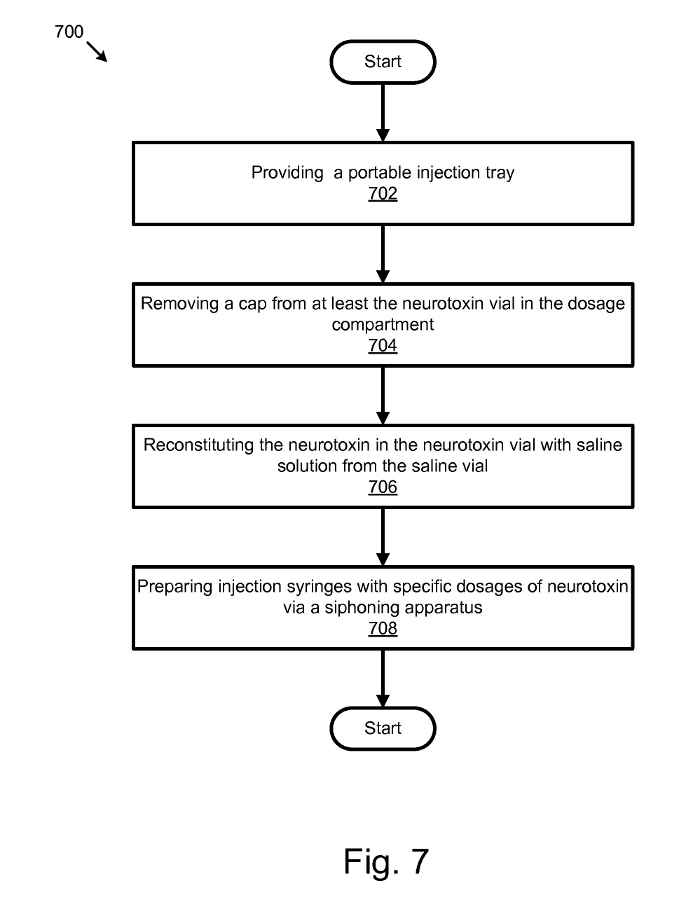

FIG. 7 is a schematic flow chart diagram of a method 700 for using a portable neurotoxin injection tray, according to one embodiment. The method 700 includes providing an injection tray at 702 that comprises the following elements: a vial section 110 comprising multiple compartments 112, 115, wherein each compartment is holding at least one neurotoxin vial 116 and at least one saline vial 113, a saline syringe section 120 holding at least one saline syringe 125, a dosage compartment 130 comprising a support member 132 and a neurotoxin vial 116 positioned on the support member 132 and situated in a tilted orientation 134, and an injection syringe section 140 comprising multiple compartments 141-144, wherein each compartment is holding at least one injection syringe 145 configured to contain a specific dosage of neurotoxin. The method 700 further includes removing a cap from at least the neurotoxin vial 116 in the dosage compartment 130 using a vial opener device 152 at 704 and reconstituting the neurotoxin vial 116 in the dosage compartment 130 by injecting saline solution from the saline vial 113 into the neurotoxin vial 116 at 706. The neurotoxin vials may be de-capped one at a time as neurotoxin is needed or multiple vials may be de-capped at once in order to prepare multiple injection dosages. Additionally, the neurotoxin vials may be de-capped while the vials are in place in the tray or the practitioner may remove the neurotoxin vials from the vial section and remove the caps from the vials in hand.

The method 700 further includes preparing the injection syringes 146-149 by filling the at least one injection syringe in each injection syringe compartment 141-144 with the specific dosage of neurotoxin from the reconstituted neurotoxin vial 116 at 708 and injecting patients with a desired quantity of neurotoxin by selecting injection syringes 146-149 from the multiple injection syringe compartments 141-144 that each contain syringes with a specific dosage. Preparing the injection syringes at 708 may include employing a siphoning apparatus to facilitate efficiently charging the injection syringes, as described above with reference to FIGS. 5D and 5E.

In the above description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object. Further, the terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Additionally, instances in this specification where one element is “coupled” to another element can include direct and indirect coupling. Direct coupling can be defined as one element coupled to and in some contact with another element. Indirect coupling can be defined as coupling between two elements not in direct contact with each other, but having one or more additional elements between the coupled elements. Further, as used herein, securing one element to another element can include direct securing and indirect securing. Additionally, as used herein, “adjacent” does not necessarily denote contact. For example, one element can be adjacent another element without being in contact with that element.

The present disclosure may be embodied in other specific forms without departing from its spirit or essential characteristics. The described embodiments are to be considered in all respects only as illustrative and not restrictive. The scope of the disclosure is, therefore, indicated by the appended claims rather than by the foregoing description. All changes which come within the meaning and range of equivalency of the claims are to be embraced within their scope.

What is claimed is:

1. A portable neurotoxin injection tray apparatus comprising:
a vial section comprising multiple compartments, wherein each compartment is capable of storing at least one neurotoxin vial or at least one saline vial;
a dosage compartment comprising a tilted support member for positioning at least one of the neurotoxin vials in a tilted orientation; and
an injection syringe section comprising multiple compartments, wherein each compartment is designated to store at least one injection syringe filled with a different dosage of neurotoxin.

2. The apparatus of claim 1, wherein the dosage compartment further comprises at least one viewing window that allows a user to see the at least one neurotoxin vial in the tilted orientation.

3. The apparatus of claim 1, further comprising a syringe cap holder.

4. The apparatus of claim 1, further comprising a vial opener device compartment.

5. The apparatus of claim 1, further comprising a sterile pads compartment.

6. The apparatus of claim 1, further comprising a medical waste disposal compartment.

7. The apparatus of claim 6, wherein the medical waste disposal compartment is detachable from the apparatus.

8. The apparatus of claim 1, further comprising a cooling pack chamber.

9. The apparatus of claim 1, further comprising a saline syringe section capable of storing at least one saline syringe, wherein the at least one saline syringe may be used to transfer saline solution from the at least one saline vial to the at least one neurotoxin vial.

10. The apparatus of claim 1, further comprising a siphoning needle compartment.

11. The apparatus of claim 1, further comprising a dosage alignment cap compartment.

12. A method for using a portable neurotoxin injection tray apparatus, the method comprising:
providing an injection tray apparatus that comprises:
a vial section comprising multiple compartments, wherein each compartment is holding at least one neurotoxin vial or at least one saline vial;
a saline syringe section holding at least one saline syringe,
a dosage compartment comprising a support member and a neurotoxin vial positioned on the support member and situated in a tilted orientation;
a vial opener device compartment; and
an injection syringe section comprising multiple injection syringe compartments, wherein each injection syringe compartment holds injection syringes containing a specific dosage of neurotoxin;
removing a cap from the neurotoxin vial in the dosage compartment using a vial opener device stored in the vial opener device compartment;
reconstituting the neurotoxin vial in the dosage compartment by injecting saline solution from one of the at least one saline vial into the neurotoxin vial; and
preparing the injection syringes by filling injection syringes with different specific dosages of neurotoxin from the reconstituted neurotoxin vial.

13. The method of claim 12, further comprising injecting patients with a desired quantity of neurotoxin by using the injection syringes with the different specific dosages from the injection syringe section.

14. The method of claim 13, wherein injecting patients comprises selecting multiple injection syringes from the multiple injection syringe compartments, and wherein the selected multiple injection syringes comprises at least one injection syringe from at least two multiple injection syringe compartments.

15. The method of claim 12, wherein before preparing the injection syringes by filling the injection syringes, the method comprises placing a dosage alignment cap on the neurotoxin vial.

16. A siphoning apparatus, comprising:
a dosage alignment cap coupled to a vial, the dosage alignment cap comprising a needle channel in an angled orientation with respect to a central axis of the vial; and
a siphoning needle insertable within the needle channel, wherein the siphoning needle is capable of extending to the bottom edge of the vial for extracting dregs, wherein the siphoning needle is attachable to an injection syringe.

17. The apparatus of claim 16, further comprising a supporting feature for maintaining the siphoning needle in the angled orientation.
